(19) United States
(12) Patent Application Publication
(10) Pub. No.: US 2013/0273062 A1
(43) Pub. Date: Oct. 17, 2013

(54) ANTIBODIES AGAINST HUMAN CD39 AND USE THEREOF

(71) Applicants: Armand Bensussan, Paris (FR); Nathalie Bonnefoy-Berard, Lyon (FR); Jean-Francois Eliaou, Montferriez sur Lez (FR); Gilles Alberici, Grezieu La Varenne (FR); Jeremy Bastid, Craponne (FR)

(72) Inventors: Armand Bensussan, Paris (FR); Nathalie Bonnefoy-Berard, Lyon (FR); Jean-Francois Eliaou, Montferriez sur Lez (FR); Gilles Alberici, Grezieu La Varenne (FR); Jeremy Bastid, Craponne (FR)

(73) Assignee: OREGA BIOTECH, Ecully (FR)

(21) Appl. No.: 13/996,097

(22) PCT Filed: Dec. 21, 2011
(86) PCT No.: PCT/EP11/73659

§ 371 Date: Jun. 20, 2013

Related U.S. Application Data
(60) Provisional application No. 61/426,041, filed on Dec. 22, 2010.

(51) Int. Cl. C07K 16/40

(52) U.S. Cl. CPC C07K 16/40
USPC 424/146.1; 435/375; 435/7.23; 530/388.26; 435/252.33; 435/254.2; 435/254.21; 435/338

(57) ABSTRACT

Disclosed are CD39 antagonists that can inhibit the immunosuppressive effect of a CD39-expressing cancerous cell, and methods of using the CD39 antagonists.

### FIELD OF THE INVENTION

The invention relates to antibodies against human CD39 for inhibiting the immunosuppressive effects of a CD39-expressing cancerous cell.

### BACKGROUND OF THE INVENTION

CD39 is an integral membrane protein with two transmembrane domains and a large extracellular region (Maliszewski et al, 1994) with nucleoside triphosphate diphosphohydrolase activity (Wang and Guidotti, 1996). In humans, CD39 is mainly expressed by regulatory T cells (Treg) but also other leukocytes. In cancers infiltrated with CD39 positive Tregs, CD39 plays a key role because it increases tumor angiogenesis and suppress the immune antitumor response by initiating the generation of adenosine (Stagg J et al, 2010).

To date, expression of CD39 by tumor cells has not been reported. Patent application WO2009/095478 describes the use of a CD39 antibody for the treatment or prevention of diseases, like cancers and infectious diseases, associated with an increased Treg activity.

However, certain cancerous pathologies are not associated with Treg activity. Thus, it would be very useful to elaborate methods and compositions for inhibiting the immunosuppressive effects of CD39-expressing cancerous cells.

The present invention aims to provide a method for inhibiting the immunosuppressive effects of CD39-expressing cancerous cells.

### SUMMARY OF THE INVENTION

The present invention relates to a CD39 antibody for inhibiting the immunosuppressive effects of a CD39-expressing cancerous cell.

In particular, the invention relates to a CD39 antibody for the treatment or prevention of cancers. Preferably, said cancer is selected in the group consisting of melanoma, haematological cancer, ovarian cancer, thyroid cancer, lung cancer, kidney cancer.

The invention also relates to a method for treating cancers, said method comprising the step of administering to said subject an effective amount of a CD39 antagonist, preferably a CD39 monoclonal antibody inhibiting the immunosuppressive effect of CD39-expressing cancerous cells, said cancerous cells being preferably selected from the group consisting of haematological cancer cells, melanoma cells, ovarian cancer cells, thyroid cancer cells, lung cancer cells, and kidney cancer cells.

### DETAILED DESCRIPTION OF THE INVENTION

### Definitions

The term “CD39” denotes the CD39 protein also named as ectonucleoside triphosphate diphosphohydrolase-1 (ENTPD1). CD39 is an ectoenzyme that hydrolases ATP/UTP and ADP/UDP to the respective nucleosides such as AMP.

The term “CD39 antibody” refers to an antibody which binds to human CD39.

According to the present invention, “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that specifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine the binding site specific to the antigenic epitope. The constant region domains of the light (CL) and heavy (CH) chains confer important biological properties such as antibody chain association, secretion, trans-placental mobility, complement binding, and binding to Fc receptors (FcR). The Fv fragment is the N-teiminal part of the Fab fragment of an immunoglobulin and consists of the variable portions of one light chain and one heavy chain. The specificity of the antibody resides in the structural complementarity between the antibody binding site and the antigenic epitope. Antibody binding sites are made up of residues that are primarily from the hypervariable or complementarity determining regions (CDRs). Occasionally, residues from nonhypervariable or framework regions (FR) influence the overall domain structure and hence the binding site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, includes six CDRs, comprising the CDR set from each of a heavy and a light chain V region. Framework Regions (FRs) refer to amino acid sequences interposed between CDRs.

According to the invention, the term “chimeric antibody” refers to an antibody which comprises a VH domain and a VL domain of a CD39 antibody from any species, preferably mouse, and a CH domain and a CL domain of a human antibody.

According to the invention, the term “humanized antibody” refers to an antibody having variable region framework and constant regions from a human antibody but retains the CDRs of a CD39 antibody from any species, preferably mouse.

The term “Fab” denotes an antibody fragment having a molecular weight of about 50,000 and antigen binding activity, in which about a half of the N-terminal side of H chain and the entire L chain, among fragments obtained by treating IgG with a protease, papaine, are bound together through a disulfide bond.

The term “F(ab′)2” refers to an antibody fragment having a molecular weight of about 100,000 and antigen binding activity, which is slightly larger than the Fab bound via a disulfide bond of the hinge region, among fragments obtained by treating IgG with a protease, pepsin.

The invention also encompasses molecules, preferably polypeptides, comprising the variable domain of the antibody of the invention, preferably the Fab of said antibody or the F(ab′)2 of said antibody.

The term “Fab′ ” refers to an antibody fragment having a molecular weight of about 50,000 and antigen binding activity, which is obtained by cutting a disulfide bond of the hinge region of the F(ab′)2.

A single chain Fv (“scFv”) polypeptide is a covalently linked VH::VL heterodimer which is usually expressed from a gene fusion including VH and VL encoding genes linked by a peptide-encoding linker. “dsFv” is a VH::VL heterodimer stabilised by a disulfide bond. Divalent and multivalent antibody fragments can form either spontaneously by association of monovalent scFvs, or can be generated by coupling monovalent scFvs by a peptide linker, such as divalent sc(Fv)2.

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites.

By “purified” and “isolated” it is meant, when referring to a polypeptide (i.e. an antibody according to the invention) or to a nucleotide sequence, that the indicated molecule is present in the substantial absence of other biological macromolecules of the same type. The term “purified” as used herein preferably means at least 75% by weight, more preferably at least 85% by weight, more preferably still at least 95% by weight, and most preferably at least 98% by weight, of biological macromolecules of the same type are present. An “isolated” nucleic acid molecule which encodes a particular polypeptide refers to a nucleic acid molecule which is substantially free of other nucleic acid molecules that do not encode the polypeptide; however, the molecule may include some additional bases or moieties which do not deleteriously affect the basic characteristics of the composition.

In the context of the invention, the term “treating” or “treatment”, as used herein, means reversing, alleviating, inhibiting the progress of, or preventing the disorder or condition to which such term applies, or one or more symptoms of such disorder or condition. A “therapeutically effective amount” is intended for a minimal amount of active agent (e.g., CD39 antibodies) which is necessary to impart therapeutic benefit to a subject. For example, a “therapeutically effective amount” to a mammal is such an amount which induces, ameliorates or otherwise causes an improvement in the pathological symptoms, disease progression or physiological conditions associated with or resistance to succumbing to a disorder.

As used herein, the term “prevention” refers to preventing the disease or condition from occurring in a subject which has not yet been diagnosed as having it.

As used herein, the term “subject” denotes a mammal, such as a rodent, a feline, a canine, and a primate. Preferably a subject according to the invention is a human.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. The terms “cancer” or “neoplasms” include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In the context of the present invention, the term “cancer” refers to melanoma, haematological cancer, ovarian cancer, thyroid cancer, lung cancer and kidney cancer.

As used herein, the term “haematological cancer” refers to cancers of blood and bone marrow, such as lymphoma, leukemia, and multiple myeloma. Preferably said haematological cancer is a lymphoma.

Leukemia refers to B-cell leukemia and T-cell leukemia and includes, but is not limited to, acute myeloid leukemia, chronic myeloid leukemia, acute lymphocytic leukemia (including, e.g., precursor B acute lymphoblastic leukemia, precursor T acute lymphoblastic leukemia, and acute biphenotypic leukemia), chronic lymphocytic leukemia (e.g., B-cell prolymphocytic leukemia), acute monocytic leukemia, acute myolegenous leukemia, chronic myelogenous leukemia, chronic granulocytic leukemia and T-cell prolymphocytic leukemia.

Lymphoma includes mantle cell lymphoma, Non-Hodgkin's lymphoma, and Hodgkin's lymphoma.

### Therapeutic Uses of CD39 Antibodies

Therefore, a first aspect of the invention provides methods and pharmaceutical compositions for inhibiting the immunosuppressive effects of a CD39-expressing cancerous cell.

The invention thus relates to a CD39 antagonist for use for inhibiting the immunosuppressive effects of a CD39-expressing cancerous cell.

The invention also relates to a method for inhibiting the immunosuppressive effects of a CD39-expressing cancerous cell which comprises the step of administering to a subject in need thereof a CD39 antagonist.

The invention also relates to a method for treating a subject suffering from a cancer, said method comprising the step of administering to said subject an effective amount of a CD39 antagonist, preferably a CD39 monoclonal antibody inhibiting the immunosuppressive effect of CD39-expressing cancerous cells, said cancerous cells being preferably selected from the group consisting of haematological cancer cells, melanoma cells, ovarian cancer cells, thyroid cancer cells, lung cancer cells, and kidney cancer cells.

The term “haematological cancer cells” refers to myeloid and/or lymphoid cells affected from cancer and includes lymphoma cells, leukemia cells and myeloma cells. Preferably, said haematological cancer cell is a lymphoma cell.

Examples of cancers to be treated include but are not limited to lung cancer, breast cancer, ovarian cancer, melanoma, skin carcinoma, liver cancer, gastric cancer, prostate cancer, pancreatic cancer, thyroid cancer, kidney cancer and lymphoma. Preferably, cancers to be treated are melanoma, haematological cancer, ovarian cancer, thyroid cancer, lung cancer and kidney cancer. Preferably, said haematological cancer is a lymphoma.

The CD39 antagonist is preferably selected in the group consisting of antibodies and chemical compounds, said antibodies and chemical compounds being able to down modulate the cell membrane expression of CD39 and/or to block or decrease CD39 ATPase/ADPase activity and/or to block or decrease cancer cells-mediated inhibition or suppression of the immune response. These CD39 antagonists significantly down modulate the cell membrane expression of CD39 and/or significantly block or decrease CD39 ATPase/ADPase activity and/or significantly block or decrease cancer cells-mediated inhibition or suppression of the immune response and/or to block or decrease cancer cells-mediated inhibition or suppression of the CD4 and/or CD8 T cell response.

The cell membrane expression of CD39 may be measured according to the protocol described in Examples 1 or 5 below.

The CD39 ATPase/ADPase activity may be measured according to the protocol described in Examples 3 or 7 below.

The cancer cells-mediated inhibition of the immune response may be measured according to the protocol described in Example 2 below.

The invention also relates to a method for identifying a subject suffering from a cancer comprising the step of determining the presence of CD39 expression on the cancerous cells of a sample from said subject, with the CD39 antibody according to the invention.

The present invention thus refers to the use of any CD39 antibody or fragment thereof, including CD39 chimeric antibodies (preferably chimeric mouse/human antibodies) or humanized CD39 antibodies, provided that said antibody inhibits the immunosuppressive effects of a CD39-expressing cancerous cell. Preferably, the CD39 antagonist is a CD39 monoclonal antibody.

Preferably, said CD39 monoclonal antibody is selected from the group consisting of BY12, BY40 and BA54g.

In a particular embodiment, said CD39 antibody is BY40. The inventors have indeed deposited a murine CD39 antibody (BY40) producing hybridoma at the Collection Nationale de Cultures de Microorganismes (CNCM, Institut Pasteur, 25 rue du Docteur Roux, 75724 Paris Cedex 15, France), in accordance with the terms of Budapest Treaty, on the 4th of January 2008. The deposited hybridoma has CNCM deposit number I-3889. Said CD39 antibody may then be obtainable from the hybridoma deposited as CNCM-I-3889.

In another embodiment, said CD39 antibody may comprise the VL chain of the antibody obtainable from hybridoma deposited as CNCM-I-3889 and the VH chain of the antibody obtainable from hybridoma deposited as CNCM-I-3889.

In another embodiment, said CD39 antibody may comprise a variable light chain (VL) comprising the CDRs of the VL chain of the antibody obtainable from hybridoma deposited as CNCM-I-3889 and a variable heavy chain (VH) comprising the CDRs of the VH chain of the antibody obtainable from hybridoma deposited as CNCM-I-3889.

In another embodiment, the invention also encompasses molecules, preferably polypeptides, comprising the variable domain of the antibody obtainable from hybridoma deposited as CNCM-I-3889, preferably the Fab of said antibody or the F(ab′)2 of said antibody.

In another embodiment of the invention, said CD39 antibody may comprise a heavy chain wherein the variable domain comprises at least one CDR having a sequence selected from the group consisting of SEQ ID NO:2 for CDR-H1, SEQ ID NO:3 for CDR-H2 and SEQ ID NO:4 for CDR-H3; and/or a light chain wherein the variable domain comprises at least one CDR having a sequence selected from the group consisting of SEQ ID NO:6 for CDR-L1, SEQ ID NO:7 for CDR-L2 and SEQ ID NO:8 for CDR-L3.

The inventors have cloned and characterized the variable domain of the light and heavy chains of said mAb BY40, and thus determined the complementarity determining regions (CDRs) domain of said antibody as described in Table 1:

TABLE 1
VH, VL and CDR domains of mAb BY40:

| MAb BY40 Domains | |
|---|---|
| VH | TRVKK PRETV KISCK ASGYT FTHYG MNWVK |
| | QAPGK GLKWM GWINT YTGEP TYADD FKGRF AFSLE |
| | ASVST AYLQI NNLKN EDTAT YFCAR RRYEG NYVFY |
| | YFDYW GQGTT LTVSS AKTTP PSVYP LAPGS AAQTN |
| | SMVTL GCLVK GYFPE QVTVT WNSGS LSSGV HTFPA |
| | VLQSD LYTLS SSVTV PS (SEQ ID NO: 1) |
| VH CDR1 | GYTFT HYG (SEQ ID NO: 2) |
| VH CDR2 | INTYT GEP (SEQ ID NO: 3) |
| VH CDR3 | ARRRY EGNYV FYYFD YWGQG TTLTV SS |
| | (SEQ ID NO: 4) |
| VL | DIQMT QSPAS LSASV GETVT ITCRA SENIY SYFSW |
| | YQQKQ GKSPQ LLVYT AKTLA EGVPS RFSGS GSGTQ |
| | FSLKI NSLQP EDFGS YYCQH HYVTP YTFGG GTKLE |
| | IKRAD AAPTV SIFPP SSEQL TSGGA SVVCF LNNFY |
| | PKDIN VKWKI DGSER QNGVL NSWTD |
| | (SEQ ID NO: 5) |
| VL CDR1 | RASEN IYSYF S (SEQ ID NO: 6) |
| VL CDR2 | TAKTL AE (SEQ ID NO: 7) |
| VL CDR3 | QHHYV TPYTF GGGTK LEIKR (SEQ ID NO: 8) |

An embodiment of the invention relates to a CD39 antibody comprising a first heavy chain CDR sequence as set forth in SEQ ID NO:2, a second heavy chain CDR sequence as set forth in SEQ ID NO:3, and a third heavy chain CDR sequence as set forth in SEQ ID NO:4; and a first light chain CDR sequence as set forth in SEQ ID NO:6, a second light chain CDR sequence as set forth in SEQ ID NO:7, and a third light chain CDR sequence as set forth in SEQ ID NO:8. In a particular embodiment, the heavy chain variable domain of said antibody has the amino acid sequence as set forth in SEQ ID NO: 1 and/or the light chain variable domain of said antibody has the amino acid sequence set forth in SEQ ID NO: 5.

Antibodies of the invention can be produced by any technique well known in the art. In particular said antibodies are produced by techniques as hereinafter described.

In another embodiment, an antibody of the invention is a chimeric antibody, preferably a chimeric mouse/human antibody. In particular, said mouse/human chimeric antibody may comprise the variable domains of an antibody obtainable from hybridoma deposited as CNCM-I-3889.

An embodiment of the invention relates to the hybridoma accessible under CNCM deposit number I-3889.

In another embodiment, an antibody of the invention is a humanized antibody. In particular, in said humanized antibody, the variable domain comprises human acceptor frameworks regions, and optionally human constant domain where present, and non-human donor CDRs, such as mouse CDRs as defined above.

The invention further provides fragments of said antibodies which include but are not limited to Fv, Fab, F(ab′)2, Fab', dsFv, scFv, sc(Fv)2 and diabodies; and multispecific antibodies formed from antibody fragments.

In another aspect, the invention relates to a polypeptide which has a sequence selected from the group consisting of SEQ ID NO: 1, SEQ ID NO:2, SEQ ID NO:3, SEQ ID NO:4, SEQ ID NO:5; SEQ ID NO: 6; SEQ ID NO:7 and SEQ ID NO:8.

A further object of the invention relates to a nucleic acid sequence encoding an antibody of the invention or a fragment thereof.

In a particular embodiment, the invention relates to a nucleic acid sequence encoding the VH domain of the antibody obtainable from hybridoma deposited as CNCM-I-3889 (BY40) or the VL domain of the antibody obtainable from hybridoma deposited as CNCM-I-3889 (BY40).

In a particular embodiment, the invention relates to a nucleic acid sequence encoding the VH domain of mAb BY40 or the VL domain of mAb BY40.

TABLE 2
Nucleic acids of VH and VL domains of mAb BY40:

| | |
|---|---|
| VH | acg cga gtg aag aag cct cga gag aca gtc aag atc tcc tgc aag gct |
| domain: | tct ggg tat acc ttc aca cac tat gga atg aac tgg gtg aag cag gct cca gga |
| | aag ggt tta aag tgg atg ggc tgg ata aac acc tac act gga gag cca aca tat |
| | gct gat gac ttc aag gga cgg ttt gcc ttc tct ttg gaa gcc tct gtc agc act gcc |
| | tat ttg cag atc aac aac ctc aaa aat gag gac acg gct aca tat ttc tgt gca aga |
| | agg aga tat gag ggt aac tac gtt ttt tac tac ttt gac tac tgg ggc caa ggc acc |
| | act ctc aca gtc tcc tca (SEQ ID NO: 9) |
| VL | gac atc cag atg act cag tct cca gcc tcc cta tct gca tct gtg gga gaa |
| domain: | act gtc acc atc aca tgt cga gca agt gaa aat att tac agt tat ttt tca tgg tat |
| | cag cag aaa cag gga aaa tct cct cag ctc ctg gtc tat act gca aaa acc tta |
| | gca gaa ggt gtg cca tca agg ttc agt ggc agt gga tca ggc aca cag ttt tct |
| | ctg aag atc aac agc ctg cag cct gaa gat ttt ggg agt tat tac tgt caa cat cat |
| | tat gtt act ccg tac acg ttc gga ggg ggg acc aag ctg gaa ata aaa cgg |
| | (SEQ ID NO: 10) |

In a particular embodiment, said CD39 antibody is BA54g. The inventors have indeed deposited the BA54g antibody producing hybridoma at the Collection Nationale de Cultures de Microorganismes (CNCM, Institut Pasteur, 25 rue du Docteur Roux, 75724 Paris Cedex 15, France), in accordance with the terms of Budapest Treaty, on the 23^{rd} of June, 2009. The deposited hybridoma has CNCM deposit number CNCM 1-4171. Said CD39 antibody may then be obtainable from the hybridoma deposited under the accession number CNCM 1-4171.

In another embodiment, said CD39 antibody may comprises the VL chain of the antibody obtainable from hybridoma deposited as CNCM-I-4171 and the VH chain of the antibody obtainable from hybridoma deposited as CNCM-I-4171.

In another embodiment, said CD39 antibody may comprise a variable light chain (VL) comprising the CDRs of the VL chain of the antibody obtainable from hybridoma deposited as CNCM-I-4171 and a variable heavy chain (VH) comprising the CDRs of the VH chain of the antibody obtainable from hybridoma deposited as CNCM-I-4171.

In another embodiment of the invention, said CD39 antibody may comprise a heavy chain wherein the variable domain comprises at least one CDR having a sequence selected from the group consisting of SEQ ID NO:12 for CDR-H1, SEQ ID NO:13 for CDR-H2 and SEQ ID NO:14 for CDR-H3; and/or a light chain wherein the variable domain comprises at least one CDR having a sequence selected from the group consisting of SEQ ID NO:16 for CDR-L1, SEQ ID NO:17 for CDR-L2 and SEQ ID NO:18 for CDR-L3.

The inventors have cloned and characterized the variable domains of the light and heavy chains of said BA54g, and thus determined the complementarity determining regions (CDRs) of said antibody as described in Table 3.

TABLE 3
VH, VL and CDR domains of mAb BA54g:

| MAb BA54g | |
|---|---|
| Domains | |
| VH | DVQLV ESGGG LVQPG GSRKL SCAAS GFTFS SFGMH |
| | WVRQA PEKGL EWVAY ISSGS SIIYY ADTVK GRFTI |
| | SRDNP KNTLF LQMTS LGSED TAMYY CARWS TTVVA |
| | TDYWG QGTTL TVS (SEQ ID NO: 11) |
| VH CDR1 | GGSRK LSCAA SGFTF SSFGM H (SEQ ID NO: 12) |
| VH CDR2 | YISSG SSIIY YADTV KG (SEQ ID NO: 13) |
| VH CDR3 | WSTTV VATDY WGQGT TLTVS (SEQ ID NO: 14) |
| VL | NIVMT QSPKS MSMSV GERVT LTCKA SENVV TYVSW |
| | YQQKP EQSPK LLIYG ASNRY TGVPD RFTGS GSATD FTLTI |
| | SSVQA EDLAD YHCGQ GYSYP YTFGG GTKLE IKR |
| | (SEQ ID NO: 15) |
| VL CDR1 | KASEN VVTYV S (SEQ ID NO: 16) |
| VL CDR2 | GASNR YT (SEQ ID NO: 17) |
| VL CDR3 | CGQGY SYPYT FGGGT KLEIK R (SEQ ID NO: 18) |

An embodiment of the invention relates to a CD39 antibody comprising a first heavy chain CDR sequence as set forth in SEQ ID NO:12, a second heavy chain CDR sequence as set forth in SEQ ID NO:13, and a third heavy chain CDR sequence as set forth in SEQ ID NO:14; and a first light chain CDR sequence as set forth in SEQ ID NO:16, a second light chain CDR sequence as set forth in SEQ ID NO:17, and a third light chain CDR sequence as set forth in SEQ ID NO:18. In a particular embodiment, the heavy chain variable domain of said antibody has the amino acid sequence as set forth in SEQ ID NO: 11 and/or the light chain variable domain of said antibody has the amino acid sequence set forth in SEQ ID NO: 15.

Said antibodies can be produced by any technique well known in the art. In particular said antibodies are produced by techniques as hereinafter described.

According to an embodiment, the antibody of the invention is a murine antibody.

In another embodiment, the antibody of the invention is a chimeric antibody, preferably a chimeric mouse/human antibody. In particular, said mouse/human chimeric antibody may comprise the variable domains of an antibody obtainable from hybridoma deposited as CNCM I-4171.

In another embodiment, the antibody of the invention is a humanized antibody. In particular, in said humanized antibody, the variable domain comprises human acceptor frameworks regions, and optionally human constant domain where present, and non-human donor CDRs of an antibody obtainable from hybridoma deposited as CNCM I-4171.

The invention further provides for fragments of said antibodies which include but are not limited to Fv, Fab, F(ab′)2, Fab′, dsFv, scFv, sc(Fv)2 and diabodies; and multispecific antibodies formed from antibody fragments.

In another aspect, the invention relates to a polypeptide which has a sequence selected from the group consisting of SEQ ID NO: 11, SEQ ID NO:12, SEQ ID NO:13, SEQ ID NO:14, SEQ ID NO:15; SEQ ID NO: 16; SEQ ID NO:17 and SEQ ID NO:18.

In a particular embodiment, the invention relates to a nucleic acid sequence encoding the VH domain of mAb BA54g or the VL domain of mAb BA54g, as detailed in Table 4.

In a particular embodiment, the invention relates to a nucleic acid sequence encoding the VH domain of the antibody obtainable from hybridoma deposited as CNCM I-4171 (SEQ ID NO:19) or the VL domain of the antibody obtainable from hybridoma deposited as CNCM I-4171 (SEQ ID NO:20).

TABLE 4
Nucleic acids of VH and VL domains of mAb BA54g:

| | |
|---|---|
| VH domain: | GATGT GCAGC TGGTG GAGTC TGGGG GAGGC TTAGT |
| | GCAGC CTGGA GGGTC CCGGA AACTC TCCTG TGCAG |
| | CCTCT GGATT CACTT TCAGT AGCTT TGGAA TGCAC |
| | TGGGT TCGTC AGGCT CCAGA GAAGG GGCTG GAGTG |
| | GGTCG CATAC ATTAG TAGTG GCAGT AGTAT TATCT |
| | ACTAT GCAGA CACAG TGAAG GGCCG ATTCA CCATC |
| | TCCAG AGACA ATCCC AAGAA CACCC TGTTC CTGCA |
| | AATGA CCAGT CTAGG GTCTG AGGAC ACGGC CATGT |
| | ATTAC TGTGC AAGAT GGAGT ACTAC GGTAG TAGCT |
| | ACAGA CTACT GGGGC CAAGG CACCA CTCTC ACAGT CTCC |
| | (SEQ ID NO: 19) |
| VL domain: | AACAT TGTAA TGACC CAATC TCCCA AATCC ATGTC |
| | CATGT CAGTA GGAGA GAGGG TCACC TTGAC CTGCA |
| | AGGCC AGTGA GAATG TGGTT ACTTA TGTTT CCTGG |
| | TATCA ACAGA AACCA GAGCA GTCTC CTAAA CTGCT |
| | GATAT ACGGG GCATC CAACC GGTAC ACTGG GGTCC |
| | CCGAT CGCTT CACAG GCAGT GGATC TGCAA CAGAT |
| | TTCAC TCTGA CCATC AGCAG TGTGC AGGCT GAAGA |
| | CCTTG CAGAT TATCA CTGTG GACAG GGTTA CAGCT |
| | ATCCG TACAC GTTCG GAGGG GGGAC CAAGC TGGAA |
| | ATAAA ACGG (SEQ ID NO: 20) |

Typically, said nucleic acid is a DNA or RNA molecule, which may be included in any suitable vector, such as a plasmid, cosmid, episome, artificial chromosome, phage or a viral vector.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence.

So, a further object of the invention relates to a vector comprising a nucleic acid of the invention.

Such vectors may comprise regulatory elements, such as a promoter, enhancer, terminator and the like, to cause or direct expression of said antibody upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like.

Any expression vector for animal cell can be used, so long as a gene encoding the human antibody C region can be inserted and expressed. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSG1 beta d2-4-(Miyaji H et al. 1990) and the like.

Other examples of plasmids include replicating plasmids comprising an origin of replication, or integrative plasmids, such as for instance pUC, pcDNA, pBR, and the like.

Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+cells, 293 cells, etc. Detailed protocols for producing such replication-defective recombinant viruses may be found for instance in WO 95/14785, WO 96/22378, U.S. Pat. No. 5,882,877, U.S. Pat. No. 6,013,516, U.S. Pat. No. 4,861,719, U.S. Pat. No. 5,278,056 and WO 94/19478.

A further object of the present invention relates to a cell which has been transfected, infected or transformed by a nucleic acid and/or a vector according to the invention.

The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA bas been “transformed”.

The nucleic acids of the invention may be used to produce an antibody of the invention in a suitable expression system. The term “expression system” means a host cell and compatible vector under suitable conditions, e.g. for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL1662, hereinafter referred to as “YB2/0 cell”), and the like.

The present invention also relates to a method of producing a recombinant host cell expressing an antibody according to the invention, said method comprising the steps of: (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said antibody. Such recombinant host cells can be used for the production of antibodies of the invention.

CD39 antibodies may be produced by any technique known in the art, such as, without limitation, any chemical, biological, genetic or enzymatic technique, either alone or in combination.

For example, CD39 antibodies can be raised according to known methods by administering the appropriate antigen or epitope to a host animal selected, e.g., from pigs, cows, horses, rabbits, goats, sheep, and mice, among others.

Various adjuvants known in the art can be used to enhance antibody production. Although antibodies useful in practicing the invention can be polyclonal, monoclonal antibodies are preferred. Monoclonal antibodies against CD39 can be prepared and isolated using any technique that provides for the production of antibody molecules by continuous cell lines in culture.

Techniques for production and isolation include but are not limited to the hybridoma technique originally described by Kohler and Milstein (1975); the human B-cell hybridoma technique (Cote et al., 1983); and the EBV-hybridoma technique (Cole et al. 1985).

Knowing the amino acid sequence of the desired antibody, one skilled in the art can readily produce said antibodies, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions. Alternatively, CD39 antibodies can be synthesized by recombinant DNA techniques well-known in the art. For example, antibodies can be obtained as DNA expression products after incorporation of DNA sequences encoding the antibodies into expression vectors and introduction of such vectors into suitable eukaryotic or prokaryotic hosts that will express the desired antibodies, from which they can be later isolated using well-known techniques.

In particular, the invention further relates to a method of producing an antibody of the invention, which method comprises the steps consisting of: (i) culturing a cell expressing a CD39 antibody under conditions suitable to allow expression of said antibody; and (ii) recovering the expressed antibody.

In another particular embodiment, the method comprises the steps of:

(i) culturing an hybridoma expressing a CD39 antibody, (e.g. the hybridoma deposited as CNCM-I-3889 or CNCM-I-4171), under conditions suitable to allow expression of antibody; and

(ii) recovering the expressed antibody.

CD39 antibodies are suitably separated from the culture medium by conventional immunoglobulin purification procedures such as, for example, protein A-Sepharose, hydroxylapatite chromatography, gel electrophoresis, dialysis, or affinity chromatography.

In a particular embodiment, a human chimeric CD39 antibody invention can be produced by obtaining nucleic sequences encoding VL and VH domains as previously described, constructing a human chimeric antibody expression vector by inserting them into an expression vector for animal cell having genes encoding human antibody CH and human antibody CL, and expressing the coding sequence by introducing the expression vector into an animal cell.

As the CH domain of a human chimeric antibody, it may be any region which belongs to human immunoglobulin, but those of IgG class are suitable and any one of subclasses belonging to IgG class, such as IgG1, IgG2, IgG3 and IgG4, can also be used. Also, as the CL of a human chimeric antibody, it may be any region which belongs to Ig, and those of kappa class or lambda class can be used.

Methods for producing chimeric antibodies involve conventional recombinant DNA and gene transfection techniques are well known in the art (See Morrison SL. et al. (1984) and patent documents U.S. Pat. No. 5,202,238; and U.S. Pat. No. 5,204,244).

A CD39 humanized antibody may be produced by obtaining nucleic acid sequences encoding CDR domains, as previously described, constructing a humanized antibody expression vector by inserting them into an expression vector for animal cell having genes encoding (i) a heavy chain constant region identical to that of a human antibody and (ii) a light chain constant region identical to that of a human antibody, and expressing the genes by introducing the expression vector into an animal cell.

The humanized antibody expression vector may be either of a type in which a gene encoding an antibody heavy chain and a gene encoding an antibody light chain exists on separate vectors or of a type in which both genes exist on the same vector (tandem type). In respect of easiness of construction of a humanized antibody expression vector, easiness of introduction into animal cells, and balance between the expression levels of antibody H and L chains in animal cells, humanized antibody expression vector of the tandem type is preferred (Shitara K et al. 1994). Examples of tandem type humanized antibody expression vector include pKANTEX93 (WO 97/10354), pEE18 and the like.

Methods for producing humanized antibodies based on conventional recombinant DNA and gene transfection techniques are well known in the art (See, e. g., Riechmann L. et al. 1988; Neuberger M S. et al. 1985). Antibodies can be humanized using a variety of techniques known in the art including, for example, CDR-grafting (EP 239,400; PCT publication WO91/09967; U.S. Pat. Nos. 5,225,539; 5,530,101; and U.S. Pat. No. 5,585,089), veneering or resurfacing (EP 592,106; EP 519,596; Padlan E A (1991); Studnicka GM et al. (1994); Roguska MA. et al. (1994)), and chain shuffling (U.S. Pat. No. 5,565,332). The general recombinant DNA technology for preparation of such antibodies is also known (see European Patent Application EP 125023 and International Patent Application WO 96/02576).

Fab can be obtained by treating an antibody which specifically reacts with human CD39 with a protease, papaine. Also, the Fab can be produced by inserting DNA encoding Fab of the antibody into a vector for prokaryotic expression system, or for eukaryotic expression system, and introducing the vector into a procaryote or eucaryote (as appropriate) to express the Fab.

F(ab′)2 can be obtained treating an antibody which specifically reacts with human CD39 with a protease, pepsin. Also, the F(ab′)2 can be produced by binding Fab′ described below via a thioether bond or a disulfide bond.

Fab′ can be obtained treating F(ab′)2 which specifically reacts with human CD39 with a reducing agent, dithiothreitol. Also, the Fab′ can be produced by inserting DNA encoding Fab′ fragment of the antibody into an expression vector for prokaryote, or an expression vector for eukaryote, and introducing the vector into a prokaryote or eukaryote (as appropriate) to perform its expression.

scFv can be produced by obtaining cDNA encoding the VH and VL domains as previously described, constructing DNA encoding scFv, inserting the DNA into an expression vector for prokaryote, or an expression vector for eukaryote, and then introducing the expression vector into a prokaryote or eukaryote (as appropriate) to express the scFv. To generate a humanized scFv fragment, a well known technology called CDR grafting may be used, which involves selecting the complementary determining regions (CDRs) from a donor scFv fragment, and grafting them onto a human scFv fragment framework of known three dimensional structure (see, e. g., WO98/45322; WO 87/02671; U.S. Pat. No. 5,859,205; U.S. Pat. No. 5,585,089; U.S. Pat. No. 4,816,567; EP0173494).

Amino acid sequence modification(s) of the antibodies described herein are contemplated. For example, it may be desirable to improve the binding affinity and/or other biological properties of the antibody. It is known that when a humanized antibody is produced by simply grafting only CDRs in VH and VL of an antibody derived from a non-human animal in FRs of the VH and VL of a human antibody, the antigen binding activity is reduced in comparison with that of the original antibody derived from a non-human animal. It is considered that several amino acid residues of the VH and VL of the non-human antibody, not only in CDRs but also in FRs, are directly or indirectly associated with the antigen binding activity. Hence, substitution of these amino acid residues with different amino acid residues derived from FRs of the VH and VL of the human antibody would reduce of the binding activity. In order to resolve the problem, in antibodies grafted with human CDR, attempts have to be made to identify, among amino acid sequences of the FR of the VH and VL of human antibodies, an amino acid residue which is directly associated with binding to the antibody, or which interacts with an amino acid residue of CDR, or which maintains the three-dimensional structure of the antibody and which is directly associated with binding to the antigen. The reduced antigen binding activity could be increased by replacing the identified amino acids with amino acid residues of the original antibody derived from a non-human animal.

Modifications and changes may be made in the structure of the antibodies of the present invention, and in the DNA sequences encoding them, and still obtain a functional molecule that encodes an antibody with desirable characteristics.

In making the changes in the amino sequences, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8) ; phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

A further object of the present invention also encompasses function-conservative variants of the antibodies of the present invention.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared.

Two amino acid sequences are “substantially homologous” or “substantially similar” when greater than 80%, preferably greater than 85%, preferably greater than 90% of the amino acids are identical, or greater than about 90%, preferably grater than 95%, are similar (functionally identical) over the whole length of the shorter sequence. Preferably, the similar or homologous sequences are identified by alignment using, for example, the GCG (Genetics Computer Group, Program Manual for the GCG Package, Version 7, Madison, Wis.) pileup program, or any of sequence comparison algorithms such as BLAST, FASTA, etc.

For example, certain amino acids may be substituted by other amino acids in a protein structure without appreciable loss of activity. Since the interactive capacity and nature of a protein define the protein's biological functional activity, certain amino acid substitutions can be made in a protein sequence, and, of course, in its DNA encoding sequence, while nevertheless obtaining a protein with like properties. It is thus contemplated that various changes may be made in the antibodies sequences of the invention, or corresponding DNA sequences which encode said antibodies, without appreciable loss of their biological activity.

It is known in the art that certain amino acids may be substituted by other amino acids having a similar hydropathic index or score and still result in a protein with similar biological activity, i.e. still obtain a biological functionally equivalent protein.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine.

Another type of amino acid modification of the antibody of the invention may be useful for altering the original glycosylation pattern of the antibody.

By “altering” is meant deleting one or more carbohydrate moieties found in the antibody, and/or adding one or more glycosylation sites that are not present in the antibody.

Glycosylation of antibodies is typically N-linked. “N-linked” refers to the attachment of the carbohydrate moiety to the side chain of an asparagine residue. The tripeptide sequences asparagine-X-serine and asparagines-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. Addition of glycosylation sites to the antibody is conveniently accomplished by altering the amino acid sequence such that it contains one or more of the above-described tripeptide sequences (for N-linked glycosylation sites).

Another type of covalent modification involves chemically or enzymatically coupling glycosides to the antibody. These procedures are advantageous in that they do not require production of the antibody in a host cell that has glycosylation capabilities for N-or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, orhydroxyproline, (e) aromatic residues such as those of phenylalanine, tyrosine, or tryptophan, or (f) the amide group of glutamine. For example, such methods are described in WO87/05330.

Removal of any carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Chemical deglycosylation is described by Sojahr H. et al. (1987) and by Edge, A S. et al. (1981). Enzymatic cleavage of carbohydrate moieties on antibodies can be achieved by the use of a variety of endo-and exo-glycosidases as described by Thotakura, N R. et al. (1987).

Another type of covalent modification of the antibody comprises linking the antibody to one of a variety of non proteinaceous polymers, eg. , polyethylene glycol, polypropylene glycol, or polyoxyalkylenes, in the manner set forth in U.S. Pat. Nos. 4,640, 835; 4,496, 689; 4,301, 144; 4,670, 417; 4,791, 192 or U.S. Pat. No. 4,179,337.

It may be also desirable to modify the antibody of the invention with respect to effector function, e.g. so as to enhance antigen-dependent cell-mediated cytotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing inter-chain disulfide bond formation in this region. The homodimeric antibody thus generated may have improved internalization capability and/or increased complement-mediated cell killing and/or antibody-dependent cellular cytotoxicity (ADCC) (Caron PC. et al. 1992; and Shopes B. 1992)

### Pharmaceutical Compositions

The invention also relates to pharmaceutical composition comprising CD39 antagonists for inhibiting the immunosuppressive effects of a CD39-expressing cancerous cell.

Therefore, CD39 antagonists, particularly CD39 antibodies, may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semI-3889solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, parenteral, intranasal, intravenous, intramuscular, subcutaneous or intraocular administration and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

To prepare pharmaceutical compositions, an effective amount of the antibody may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

A CD39 antagonist can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

CD39 antibodies may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds formulated for parenteral administration, such as intravenous or intramuscular injection, other pharmaceutically acceptable forms include, e.g. tablets or other solids for oral administration; time release capsules; and any other form currently used.

In certain embodiments, the use of liposomes and/or nanoparticles is contemplated for the introduction of antibodies into host cells. The formation and use of liposomes and/or nanoparticles are known to those of skill in the art.

Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles that meet these requirements are contemplated for use in the present invention, and such particles may be are easily made.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core. The physical characteristics of liposomes depend on pH, ionic strength and the presence of divalent cations.

Antibodies of the invention can be produced and/or modified by any technique known in the art, as above described.

### FIGURES

FIG. 1: Expression of CD39 by melanoma cells. Melanoma cell lines (HN11, SK-MEL-5 and SK-MEL-28) and melanoma cells from a primary tumor (MCM) were stained with CD39 antibody (BA54g or clone A1, Ebioscience) and CD39 expression was analyzed by flow cytometry. The percentage of CD39 positive tumor cells within the population is indicated. MCM, SK-MEL-5 and SK-MEL-28 express CD39 at their cell surface.

FIG. 2: Immunosuppressive activity of melanoma cells. 1×10⁵ activated CD8 (A) or activated CD4 (B) T cells from healthy donors' blood were incubated alone or in the presence of melanoma cell line (SK-MEL-5) at 1:1 or 2:1 ratios (SK-MEL-5:T cells). Anti-CD3 mAb (1 μg/mL, clone UCHT1) was previously immobilized on cell culture plates (T cell activation). Anti-CD28 mAb (1 μg/mL, clone CD28.2) was added at the beginning of the culture. After 4 days of culture, incorporation of thymidine (18h) was performed to measure CD8 and CD4 T cells' proliferation. Each condition was made in triplicate.

FIG. 3: CD39 (BA54g) antibody inhibits CD39 ATPase activity of melanoma cells. 1×10⁵ melanoma cells (SK-MEL-5) were cultured in complete RPMI medium alone or in presence of BA54g mAb for 16 h. Cells were then washed three times in phosphate free buffer (10 mM glucose, 20 mM Hepes, 5 mM KCl, 120 mM NaCl, 2 mM CaCl2) and resuspended in the same buffer supplemented with 100 μM ATP for 15 min. Phosphate concentration in supernatants was measured after addition of Malachite green/polyvinylalcohol/ammonium mobylate solution for 20 min by a spectrophotometer at 620 nm and compared against a standard curve. Experiment was performed in triplicate.

FIG. 4: CD39 antibody (BY40) restores T cell responses against melanoma cells. 5×10⁴ CFSE-labelled PBMC were cultured in the presence of 5×10³ irradiated (100 Gy) SK-MEL-5 (NT). POM-1 (100 or 250 μM), BY40 or control isotype antibody D6212 (5 μg/mL) were added at the beginning of the culture and after 2 days of culture. After 5 days of culture, proliferation of CFSE-labelled CD4 (A) and CD8 (B) T cells was analysed by flow cytometry. Experiment was performed in duplicate and results are representative of 2 independent experiments.

FIG. 5: Expression of CD39 by lymphoma cells. Lymphoma cell lines (BL41, burkitt lymphoma; B 104, B cell lymphoma; Ramos, Burkitt lymphoma) were stained with CD39 antibody (clone A1; Ebioscience) and CD39 expression was analyzed by flow cytometry. Results are representative of four experiments. BL41, B104 and Ramos express CD39 at their cell surface.

FIG. 6: ATPAse activity of lymphoma cells. (A) 5×10⁴ lymphoma cells expressing CD39 (Ramos, BL41) or not (BJAB) were cultured in phosphate free buffer supplemented with 100 μM ATP for 15 min. Phosphate concentration in supernatants was measured after addition of Malachite green/polyvinylalcohol/ammonium mobylate solution for 10 min using a spectrophotometer at 620 nm. Experiment was performed in triplicate. (B) 5×10⁴ lymphoma cells expressing CD39 (BL41 and B104) were cultured in complete RPMI medium for 30 min with 10 μM ATP. The concentration of “unhydrolysed” extracellular ATP was determined using the ATPlite luminescence ATP Detection Assay System (Perkin Elmer). Results are the mean of 2 independent experiments.

FIG. 7: CD39 antibodies inhibit CD39 ATPase activity of lymphoma cells. 1×10⁵ lymphoma cells (Ramos) were cultured in complete RPMI medium alone or in presence of BA54g, BY12 or BY40 mAbs, at the indicated concentrations, for 16 h. Cells were then washed three times in phosphate free buffer (10 mM glucose, 20 mM Hepes, 5 mM KCl, 120 mM NaCl, 2 mM CaCl2) and resuspended in the same buffer supplemented with 100 μM ATP for 15 min. Phosphate concentration in supernatants was measured after addition of Malachite green/polyvinylalcohol /ammonium mobylate solution for 20 min by a spectrophotometer at 620 nm and compared against a standard curve. Results are the mean of two independent experiments.

FIG. 8: Expression and activity of CD39 on ovarian cancer cells. (A) Ovarian cancer cells (OAW42) were stained with CD39 antibody (clone A1, Ebiosciences) and CD39 expression was analysed by flow cytometry. Results are representative of two independent experiments. (B) 5×10⁴ OAW42 cells were cultured in complete RPMI medium for 30 min with 10 μM ATP. The concentration of “unhydrolysed” extracellular ATP was determined using the ATPlite luminescence ATP Detection Assay System.(Perkin Elmer) Experiment was performed in triplicate.

FIG. 9: CD39 Expression on a panel of normal and cancer tissues. Multiple organ cancer and normal tissue Microarray (MC5002) from US Biomax, Inc. were used to analyze expressions of CD39. MC5002 contained 500 cores with 18 of most common types of cancer (20 -25 cases/type) and normal controls (5 cases/type). The tissues were formalin fixed, paraffin embedded. Array sections were mounted on the positive charged Fisher SuperFrost glass slide which is suitable for antigen detection. Two CD39 antibodies were evaluated for IHC suitability and the CD39 antibody from Abcam (clone 22A9) were selected. IHC staining was then performed using this CD39 antibody at 1.52 μg/mL (clone 22A9, Abcam). Manual scoring of intensity of staining was performed. The intensity (Strength, 0-3) of CD39 staining was scored as negative (0), possible positive (0.5), weak (1), moderate (2), or strong (3). Results for normal or cancer tissues from lymph node (A), thyroid (B), lung (C) or kidney (D) are shown. Each dot represents one sample.

### EXAMPLES

### Example 1

### Expression of CD39 by Melanoma Cells

### Protocol:

Melanoma cell lines (HN11, SK-MEL-5 and SK-MEL-28) and melanoma cells from a primary tumor (MCM) were stained with CD39 antibody (BA54g or clone A1 from Ebioscience) and CD39 expression was analyzed by flow cytometry. The percentage of CD39 positive tumor cells within the population was then evaluated.

### Results:

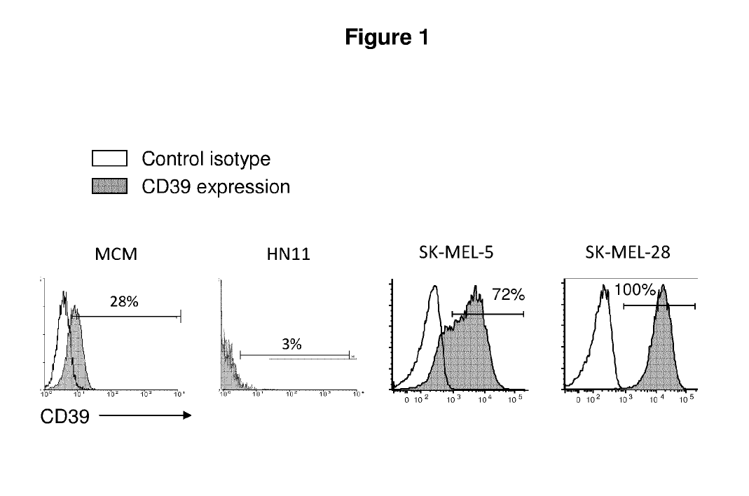

CD39 is expressed by melanoma tumor cells. CD39 is an ectonucleotidase that converts extra-cellular ATP generated in sites of immune activation, leading to adenosine generation, an inhibitor of cell proliferation. It has been reported that CD39 is expressed at the cell surface of human Tregs as well as other leukocytes (Stagg J, Oncogene 2010) but not at the surface of tumor cells. Here we used the CD39 antibody BA54g as well as a commercially available one (clone A1 from Ebioscience) to examine human CD39 expression at the cell surface of human melanoma cells. As shown in FIG. 1, melanoma cells from a human primary tumor (MCM) and from SK-MEL-5 and SK-MEL-28 cell lines display membranous expression of CD39, while another cell line (HN11) is negative for CD39. Thus, melanoma tumor cells can express CD39 at their cell surface.

### Example 2

### Activity of CD39 Positive Melanoma Cells Towards Effector T Cells

### Protocol:

1×10⁵ activated CD8 (A) or activated CD4 (B) T cells from healthy donors' blood were incubated alone or in the presence of melanoma cell line (SK-MEL-5) at 1:1 or 2:1 ratios (SK-MEL-5:T cells). Anti-CD3 mAb (1 μg/mL, clone UCHT1) was previously immobilized on cell culture plates (T cell activation). Anti-CD28 mAb (1 μg/mL, clone CD28.2) was added at the beginning of the culture. After 4 days of culture, incorporation of thymidine (18h) was performed to measure CD8 and CD4 T cells' proliferation. Each condition was made in triplicate.

### Results:

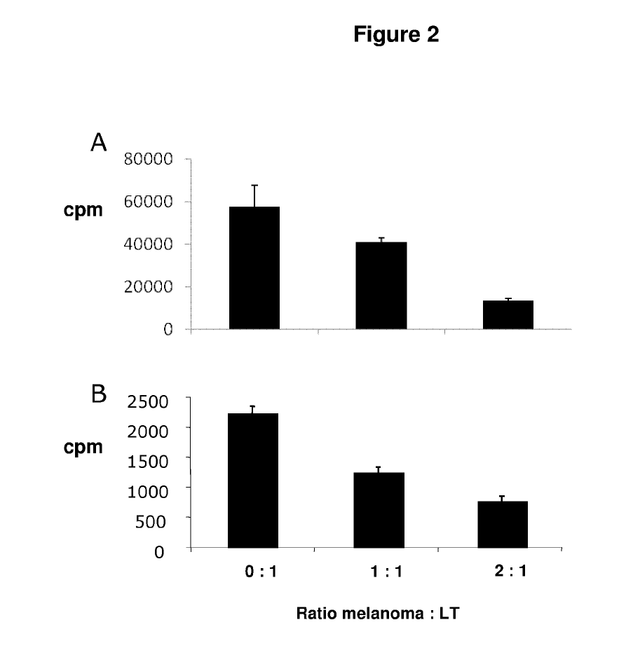

CD39 positive melanoma cells are suppressive towards CD4 and CD8 effector T cells. As CD39 participates to the generation of adenosine that is a potent immunosuppressor, we speculated that CD39 positive melanoma cells would inhibit CD4 and CD8 activated T cell proliferation. As shown in FIG. 2, SK-MEL-5 cells indeed inhibit CD4 and CD8 effector T cells proliferation in a dose dependant manner. Thus, CD39 positive melanoma cells exert a potent immunosuppressive activity which might affect the antitumor response in vivo.

### Example 3

### Inhibition of Melanoma-CD39 Enzymatic Activity by BA54g

Protocol: 1×10⁵ melanoma cells (SK-MEL-5) were cultured in complete RPMI medium alone or in presence of BA54g mAb for 16 h. Cells were then washed three times in phosphate free buffer (10 mM glucose, 20 mM Hepes, 5 mM KCl, 120 mM NaCl, 2 mM CaCl2) and resuspended in the same buffer supplemented with 100 μM ATP for 15 min Phosphate concentration in supernatants was measured after addition of Malachite green/polyvinylalcohol/ammonium mobylate solution for 20 min by a spectrophotometer at 620 nm and compared against a standard curve. Experiment was performed in triplicate.

### Results:

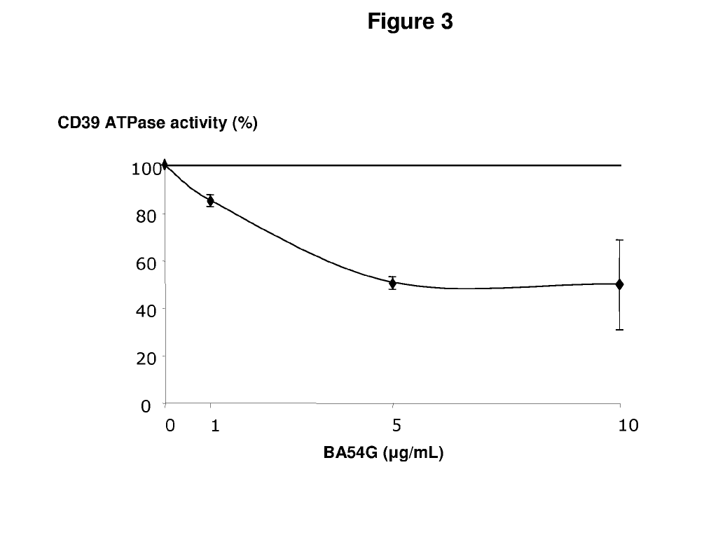

Inhibition of melanoma-CD39 enzymatic activity by CD39 monoclonal antibody (mAb) BA54g. In order to alleviate the adenosine-mediated immunosuppression by melanoma cells, we cultured CD39 positive SK-MEL-5 cells in the presence of CD39 mAb BA54g and measured CD39 enzymatic activity. As shown in FIG. 3, BA54g inhibits CD39 enzymatic activity in a dose dependant manner, suggesting that CD39 mAbs might be useful tools to abrogate the generation of adenosine by melanoma cells and to restore an efficient antitumor response.

### Example 4

### Inhibition of Melanoma Cell—Mediated Suppression by BY40

### Protocol:

5×10⁴ CFSE-labelled PBMC were cultured in the presence of 5×10³ irradiated (100 Gy) SK-MEL-5 (NT). POM-1 (100 or 250 μM), BY40 or control isotype antibody D6212 (5 μg/mL) were added at the beginning of the culture and after 2 days of culture. After 5 days of culture, proliferation of CFSE-labelled CD4 (A) and CD8 (B) T cells was analysed by flow cytometry. Experiment was performed in duplicate and results are representative of 2 independent experiments.

### Results:

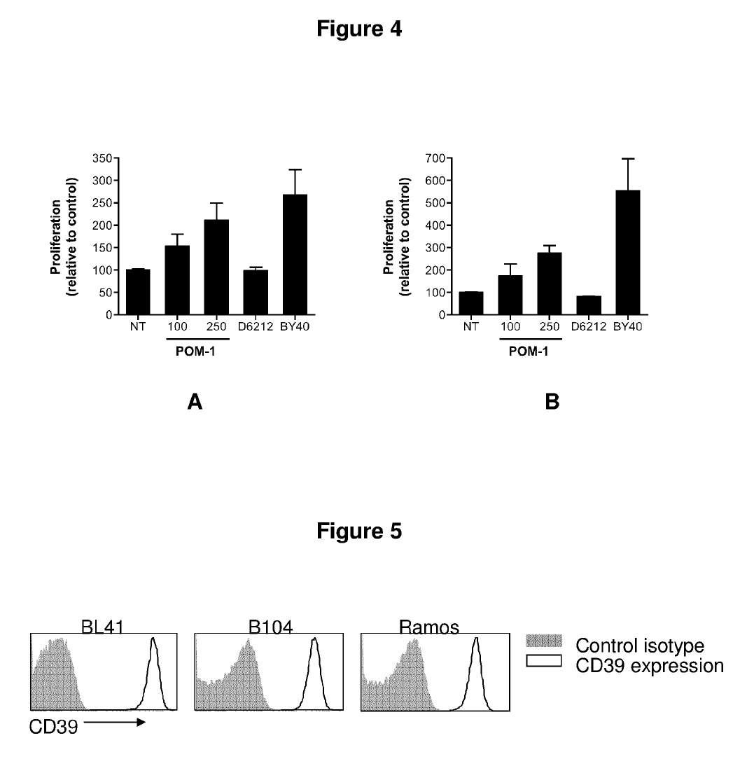

CD39 antibody (BY40) restores T cell responses against melanoma cells. CD39 positive melanoma cells are suppressive towards CD4 and CD8 T cells. As CD39 blocking antibodies inhibit CD39 enzymatic activity (FIG. 3), we analysed whether inhibition of CD39 at the cell surface of melanoma cells restores T cell proliferation. For that purpose, we co-cultured T lymphocytes and irradiated melanoma cells in the presence of CD39 mAb BY40 or a synthetic inhibitor of CD39, POM-1 (sodium metatungstate). As shown in FIG. 4, BY40 and POM-1 increased CD4 and CD8 T cell proliferation. Thus, inhibition of effector T cells by CD39-expressing melanoma cells can be abrogated by CD39 blocking mAbs.

### Example 5

### Expression of CD39 by Lymphoma Tumor Cells

### Protocol:

Lymphoma cell lines (BL41, burkitt lymphoma; B104, B cell lymphoma; Ramos, Burkitt lymphoma) were stained with CD39 antibody (clone A1; Ebioscience) and CD39 expression was analyzed by flow cytometry. Results are representative of four experiments.

### Results:

CD39 is expressed by lymphoma tumor cells. In order to assess whether lymphoma cells might also express CD39, we measured CD39 expression at the cell surface of various lymphoma tumor cell lines by flow cytometry using the CD39 antibody A1 (Ebioscience). As shown in FIG. 5, the Burkitt lymphoma cell lines BL41 and Ramos as well as the B cell lymphoma cell line B104 all express CD39 at their cell surface. Thus, lymphoma cells can express CD39 which might participate to suppress the immune system response.

### Example 6

### ATPase Activity of Lymphoma Cells

### Protocol:

(A) 5×10⁴ lymphoma cells expressing CD39 (Ramos, BL41) or not (BJAB) were cultured in phosphate free buffer supplemented with 100 μM ATP for 15 min. Phosphate concentration in supernatants was measured after addition of Malachite green/polyvinylalcohol/ammonium mobylate solution for 10 min using a spectrophotometer at 620 nm. Experiment was performed in triplicate. (B) 5×10⁴ lymphoma cells expressing CD39 (BL41 and B104) were cultured in complete RPMI medium for 30 min with 10 μM ATP. The concentration of “unhydrolysed” extracellular ATP was determined using the ATPlite luminescence ATP Detection Assay System (Perkin Elmer).

### Results:

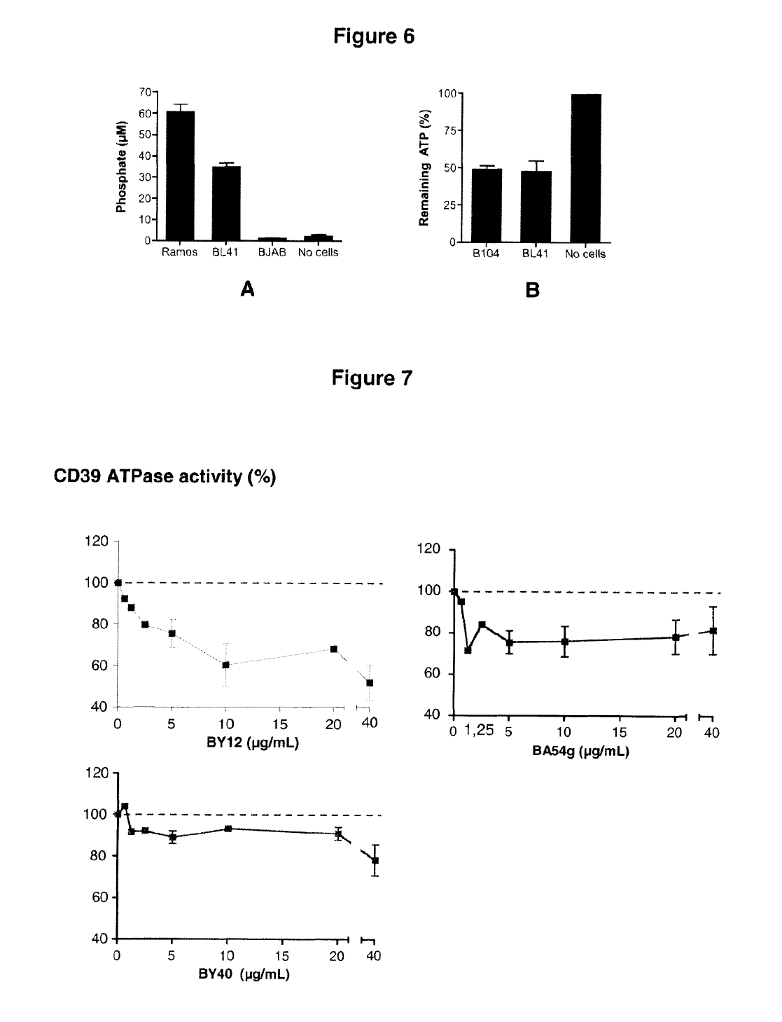

CD39 positive lymphoma cells, but not CD39 negative lymphoma cells, exert an ATPase activity. In order to assess the enzymatic activity of CD39 at the surface of Ramos, BL41 and B 104 cell lines, we measured their capacity to metabolize ATP, either by measuring phosphate concentration in cell supernatant or by measuring the remaining ATP. As shown in FIG. 6, the CD39 positive cell lines (Ramos, BL41 and B104), but not the negative one (BJAB), had the ability to hydrolyse ATP. Thus CD39 positive lymphoma cells exert an ATPase activity and might suppress antitumor responses.

### Example 7

### Inhibition of Lymphoma-CD39 Enzymatic Activity by BA54g, BY12 and BY40

### Protocol:

1×10⁵ lymphoma cells (Ramos) were cultured in complete RPMI medium alone or in presence of BA54g, BY12 or BY40 mAbs, at the indicated concentrations, for 16 h. Cells were then washed three times in phosphate free buffer (10 mM glucose, 20 mM Hepes, 5 mM KC1, 120 mM NaCl, 2 mM CaCl2) and resuspended in the same buffer supplemented with 100 μM ATP for 15 min. Phosphate concentration in supernatants was measured after addition of Malachite green/polyvinylalcohol /ammonium mobylate solution for 20 min by a spectrophotometer at 620 nm and compared against a standard curve. Results are the mean of two independent experiments.

### Results:

Inhibition of lymphoma-CD39 enzymatic activity by CD39 mAbs BA54g, BY12 and BY40. In order to test whether CD39 mAbs might be useful to inhibit CD39-mediated immunosupression by lymphoma cells we cultured the CD39 positive Ramos cell line with increasing doses of 3 CD39 mAbs. As shown in FIG. 7, the three antibodies BA54g, BY12 and BY40 inhibited CD39 activity in a dose dependant manner. Altogether these results demonstrate that CD39 is expressed and active at the surface of some cancer cells (e.g., melanoma and lymphoma). CD39 activity participates to the generation of adenosine which leads to the decreased response of the immune system with an inhibition of the antitumor response. In conclusion we show here that CD39 mAbs BA54g, BY12 and BY40 are able to decrease CD39 enzymatic activity and are thus promising tools to block tumor-CD39 mediated immunosuppression.

### Example 8

### Expression and Activity of CD39 on Ovarian Cancer Cells

### Protocol:

(A) Ovarian cancer cells (OAW42) were stained with CD39 antibody (clone A1, Ebiosciences) and CD39 expression was analysed by flow cytometry. Results are representative of two independent experiments. (B) 5×10⁴ OAW42 cells were cultured in complete RPMI medium for 30 min with 10 μM ATP. The concentration of “unhydrolysed” extracellular ATP was determined using the ATPlite luminescence ATP Detection Assay System (Perkin Elmer). Experiment was performed in triplicate.

### Results:

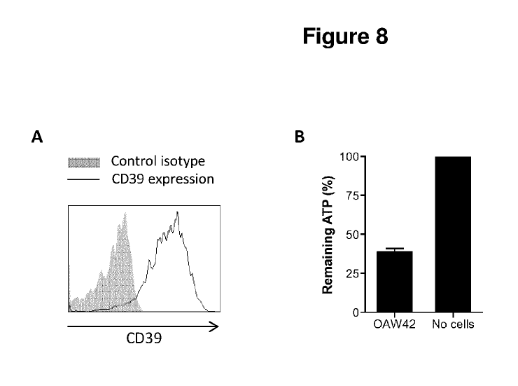

Ovarian cancer cells express functional CD39. In order to assess whether ovarian cancer cells might also expressed CD39, we measured CD39 expression at the cell surface of the ovarian cancer cell line OAW42 by flow cytometry using the CD39 mAb A1 (Ebiosciences). As shown in FIG. 8, OAW42 cells expressed CD39 at their cell surface. Furthermore, we measured their capacity to hydrolyze ATP by measuring ATP remaining in cell supernatant after culture of cells with ATP. As shown in FIG. 8, OAW42 cells degraded about 60% of ATP after 30 min of culture. Thus, ovarian cancer cells express a functional CD39 enzyme and might thus participate to suppress antitumor responses through the degradation of ATP and generation of adenosine.

### Example 9

### Expression of CD39 on a Panel of Normal and Cancer Tissues

### Protocol:

Multiple organ cancer and normal tissue Microarray (MC5002) from US Biomax, Inc. were used to analyze expressions of CD39. MC5002 contained 500 cores with 18 of most common types of cancer (20 -25 cases/type) and normal controls (5 cases/type). The tissues were formalin fixed, paraffin embedded. Array sections were mounted on the positive charged Fisher SuperFrost glass slide which is suitable for antigen detection. IHC staining was performed using CD39 antibody at 1.52 μg/mL (clone 22A9, Abeam). Manual scoring of intensity of staining was performed. The intensity (Strength, 0-3) of CD39 staining was scored as negative (0), possible positive (0.5), weak (1), moderate (2), or strong (3). Results for normal or cancer tissues from lymph node (A), thyroid (B), lung (C) or kidney (D) are shown. Each dot represents one sample.

### Results:

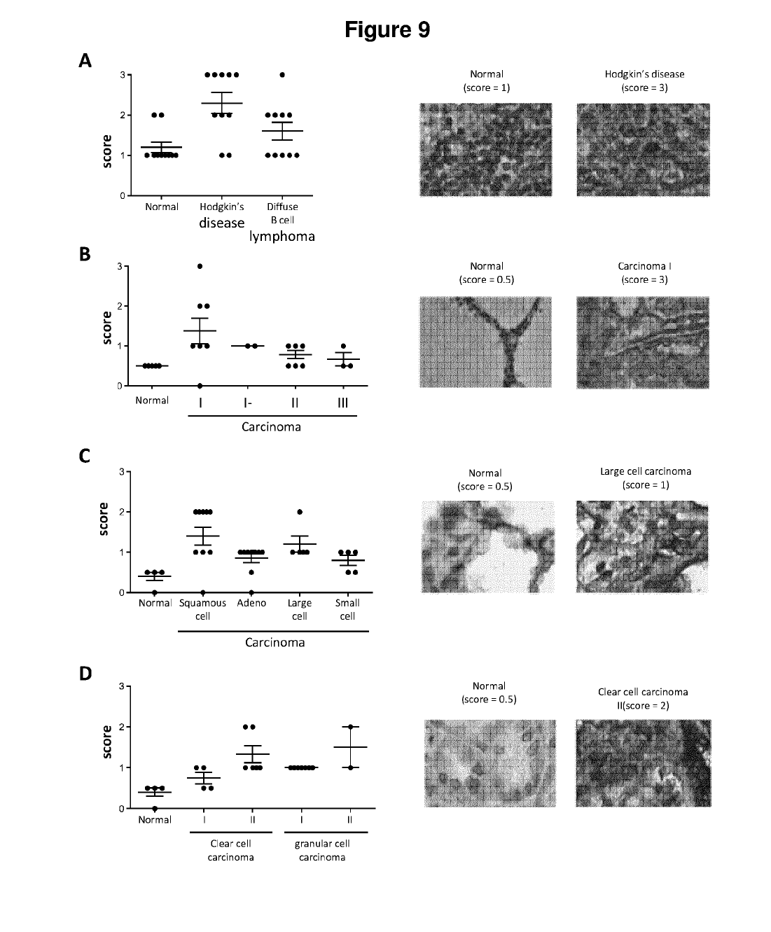

CD39 is expressed by tumor cells in various human cancers. In order to assess CD39 expression in a large panel of cancer, we analyzed CD39 expression in cancer and normal tissue Microarray. As shown on FIG. 9, CD39 staining scored as moderate to strong on many cancer tissues including lymphoma cells or carcinoma from thyroid lung or kidney. For these tissues, CD39 staining is higher in cancer samples compared to that of normal tissues.

1. A method for inhibiting the immunosuppressive effects of a CD39-expressing cancerous cell, comprising contacting said cancerous cell with a CD39 antagonist.

2. The method according to claim 1, wherein said cancerous cell is selected from haematological cancer cells, melanoma cells, ovarian cancer, thyroid cancer, lung cancer, kidney cancer.

3. The method according to claim 1, wherein said antagonist is selected from the group consisting of antibodies and chemical compounds, said antibodies and chemical compounds having activity that can down regulate the cell membrane expression of CD39, block or decrease CD39 ATPase/ADPase activity, block or decrease cancer cells-mediated inhibition or suppression of the immune antitumor response, or block or decrease cancer cells-mediated inhibition or suppression of the CD4 and/or CDS T cell response.

4. The method according to claim 3, wherein said antagonist is a CD39 monoclonal antibody.

5. The method according to claim 4, wherein said monoclonal antibody comprises:
a heavy chain wherein the variable domain comprises at least one CDR having a sequence selected from the group consisting of SEQ ID NO:2 for CDR-H1, SEQ ID NO:3 for CDR-H2 and SEQ ID NO:4 for CDR-H3; and/or
a light chain wherein the variable domain comprises at least one CDR having a sequence selected from the group consisting of SEQ ID NO:6 for CDR-L1, SEQ ID NO:7 for CDR-L2 and SEQ ID NO:S for CDR-L3.

6. The method according to claim 4, wherein said monoclonal antibody comprises:
a heavy chain wherein the variable domain comprises at least one CDR having a sequence selected from the group consisting of SEQ ID NO: 12 for CDR-H1, SEQ ID NO: 13 for CDR-H2 and SEQ ID NO: 14 for CDR-H3; and/or
a light chain wherein the variable domain comprises at least one CDR having a sequence selected from the group consisting of SEQ ID NO: 16 for CDR SEQ ID NO:17 for CDR-L2 and SEQ ID NO;18 for CDR-L3.

7. The method according to claim 4, wherein said monoclonal antibody is selected from the group consisting of BYI2, BY40 and BA54g.

8. The method according to claim 4, wherein said monoclonal antibody is selected from the group consisting of:
the monoclonal antibody produced by the hybridoma cell line deposited with the CNCM under the accession number I-3889; and
the monoclonal antibody produced by the hybridoma cell line deposited with the CNCM under the accession number CNCM I-4171.

9. A method of treating cancer comprising administering to a subject in need of treatment an effective amount of a CD39 antagonist selected from the group consisting of antibodies and chemical compounds.

10. The method according to claim 9, wherein said cancer is selected from the group consisting of melanoma, haematological cancer, ovarian cancer, thyroid cancer, lung cancer, and kidney cancer.

11. The method according to claim 10 wherein said cancer is selected from the group consisting of haematological cancer and melanoma.

12. The method according to claim 11, wherein said CD39 antagonist comprises a monoclonal antibody is selected from the group consisting of BY12, BY4O and BA54g.

13. A method for identifying a subject suffering from a cancer comprising the step of determining the presence of CD39 expression on the cancerous cells of a sample from said subject, using the CD39 antibody according to claim 18.

14. The method according to claim 9, wherein said CD39 antagonist comprises a monoclonal antibody comprising:
a heavy chain wherein the variable domain comprises at least one CDR having a sequence selected from the group consisting of SEQ ID NO:2 for CDR-H1, SEQ ID NO:3 for CDR-H2 and SEQ ID NO:4 for CDR-H3; and/or
a light chain wherein the variable domain comprises at least one CDR having a sequence selected from the group consisting of SEQ ID NO:6 for CDR-L1, SEQ ID NO:7 for CDR-L2 and SEQ IDNO:S for CDR-L3.

15. The method according to claim 9, wherein said CD39 antagonist comprises a monoclonal antibody comprising:
a heavy chain wherein the variable domain comprises at least one CDR having a sequence selected from the group consisting of SEQ ID NO: 12 for CDR-H1, SEQ ID NO: 13 for CDR-H2 and SEQ ID NO: 14 for CDR-H3: and/or
a light chain wherein the variable domain comprises at least one CDR having a sequence selected from the group consisting of SEQ ID NO: 16 for CDR -L1, SEQ ID NO:17 for CDR-L2 and SEQ ID NO:18 for CDR-L3.

16. The method according to claim 9, wherein said CD39 antagonist comprises a monoclonal antibody selected from the group consisting of:
the antibody produced by the hybridoma cell line deposited with the CNCM under the accession number I-3889; and
the antibody produced by the hybridoma cell line deposited with the CNCM under the accession number CNCM I-4171.

17. A hybridoma deposited with the CNCM having accession number I-3889 or I-4171.

18. An isolated CD39 monoclonal antibody produced by the hybridoma of claim 17.

19. The monoclonal antibody according to claim 18, or a CD39 binding fragment thereof, comprising:
a heavy chain wherein the variable domain comprises at least one CDR having a sequence selected from the group consisting of SEQ ID NO:2 for CDR-H1, SEQ ID NO:3 for CDR-H2 and SEQ ID NO:4 for CDR-H3; and/or
a light chain wherein the variable domain comprises at least one CDR having a sequence selected from the group consisting of SEQ ID NO:6 for CDR-L1, SEQ ID NO:7 for CDR-L2 and SEQ IDNO:S for CDR-L3.

20. The monoclonal antibody according to claim 18, or a CD39 binding fragment thereof, comprising:
a heavy chain wherein the variable domain comprises at least one CDR having a sequence selected from the group consisting of SEQ ID NO: 12 for CDR-H1, SEQ ID NO: 13 for CDR-H2 and SEQ ID NO: 14 for CDR-H3; and/or
a light chain wherein the variable domain comprises at least one CDR having a sequence selected from the group consisting of SEQ ID NO: 16 for CDR -L1 SEQ ID NO:17 for CDR-L2 and SEQ ID NO:18 for CDR-L3.


SEQUENCE LISTING (20 sequences)

SEQ ID NO: 1 - protein, 187 aa, Mus musculus
TRVKKPRETVKISCKASGYTFTHYGMNWVKQAPGKGLKWMGWINTYTGEPTYADDFKGRFAFSLEASVSTAYLQINNLKNEDTATYFCARRRYEGNYVFYYFDYWGQGTTLTVSSAKTTPPSVYPLAPGSAAQTNSMVTLGCLVKGYFPEQVTVTWNSGSLSSGVHTFPAVLQSDLYTLSSSVTVPS

SEQ ID NO: 2 - protein, 8 aa, Mus musculus
GYTFTHYG

SEQ ID NO: 3 - protein, 8 aa, Mus musculus
INTYTGEP

SEQ ID NO: 4 - protein, 27 aa, Mus musculus
ARRRYEGNYVFYYFDYWGQGTTLTVSS

SEQ ID NO: 5 - protein, 165 aa, Mus musculus
DIQMTQSPASLSASVGETVTITCRASENIYSYFSWYQQKQGKSPQLLVYTAKTLAEGVPSRFSGSGSGTQFSLKINSLQPEDFGSYYCQHHYVTPYTFGGGTKLEIKRADAAPTVSIFPPSSEQLTSGGASVVCFLNNFYPKDINVKWKIDGSERQNGVLNSWTD

SEQ ID NO: 6 - protein, 11 aa, Mus musculus
RASENIYSYFS

SEQ ID NO: 7 - protein, 7 aa, Mus musculus
TAKTLAE

SEQ ID NO: 8 - protein, 20 aa, Mus musculus
QHHYVTPYTFGGGTKLEIKR

SEQ ID NO: 9 - DNA, 345 bp, Mus musculus
acgcgagtgaagaagcctcgagagacagtcaagatctcctgcaaggcttctgggtataccttcacacactatggaatgaactgggtgaagcaggctccaggaaagggtttaaagtggatgggctggataaacacctacactggagagccaacatatgctgatgacttcaagggacggtttgccttctctttggaagcctctgtcagcactgcctatttgcagatcaacaacctcaaaaatgaggacacggctacatatttctgtgcaagaaggagatatgagggtaactacgttttttactactttgactactggggccaaggcaccactctcacagtctcctca

SEQ ID NO: 10 - DNA, 324 bp, Mus musculus
gacatccagatgactcagtctccagcctccctatctgcatctgtgggagaaactgtcaccatcacatgtcgagcaagtgaaaatatttacagttatttttcatggtatcagcagaaacagggaaaatctcctcagctcctggtctatactgcaaaaaccttagcagaaggtgtgccatcaaggttcagtggcagtggatcaggcacacagttttctctgaagatcaacagcctgcagcctgaagattttgggagttattactgtcaacatcattatgttactccgtacacgttcggaggggggaccaagctggaaataaaacgg

SEQ ID NO: 11 - protein, 118 aa, Mus musculus
DVQLVESGGGLVQPGGSRKLSCAASGFTFSSFGMHWVRQAPEKGLEWVAYISSGSSIIYYADTVKGRFTISRDNPKNTLFLQMTSLGSEDTAMYYCARWSTTVVATDYWGQGTTLTVS

SEQ ID NO: 12 - protein, 21 aa, Mus musculus
GGSRKLSCAASGFTFSSFGMH

SEQ ID NO: 13 - protein, 17 aa, Mus musculus
YISSGSSIIYYADTVKG

SEQ ID NO: 14 - protein, 20 aa, Mus musculus
WSTTVVATDYWGQGTTLTVS

SEQ ID NO: 15 - protein, 108 aa, Mus musculus
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKR

SEQ ID NO: 16 - protein, 11 aa, Mus musculus
KASENVVTYVS

SEQ ID NO: 17 - protein, 7 aa, Mus musculus
GASNRYT

SEQ ID NO: 18 - protein, 21 aa, Mus musculus
CGQGYSYPYTFGGGTKLEIKR

SEQ ID NO: 19 - DNA, 354 bp, Mus musculus
gatgtgcagctggtggagtctgggggaggcttagtgcagcctggagggtcccggaaactctcctgtgcagcctctggattcactttcagtagctttggaatgcactgggttcgtcaggctccagagaaggggctggagtgggtcgcatacattagtagtggcagtagtattatctactatgcagacacagtgaagggccgattcaccatctccagagacaatcccaagaacaccctgttcctgcaaatgaccagtctagggtctgaggacacggccatgtattactgtgcaagatggagtactacggtagtagctacagactactggggccaaggcaccactctcacagtctcc

SEQ ID NO: 20 - DNA, 324 bp, Mus musculus
aacattgtaatgacccaatctcccaaatccatgtccatgtcagtaggagagagggtcaccttgacctgcaaggccagtgagaatgtggttacttatgtttcctggtatcaacagaaaccagagcagtctcctaaactgctgatatacggggcatccaaccggtacactggggtccccgatcgcttcacaggcagtggatctgcaacagatttcactctgaccatcagcagtgtgcaggctgaagaccttgcagattatcactgtggacagggttacagctatccgtacacgttcggaggggggaccaagctggaaataaaacgg